(19) United States
(12) Patent Application Publication
(10) Pub. No.: US 2005/0059907 A1
(43) Pub. Date: Mar. 17, 2005

(54) Urine sample collector for women

(76) Inventor: Mari Takahashi, Noshiro (JP)

Correspondence Address: AKIKO KOBAYASHI, 2313 CAMINO ROBLEDO, CARLSBAD, CA, 92009, US

(21) Appl. No.: 10/973,962

(22) Filed: Oct. 25, 2004

(30) Foreign Application Priority Data
Apr. 13, 2001 (JP) 2001-099836

(51) Int. Cl. A61B 5/00

(52) U.S. Cl. 600/574

(57) ABSTRACT

The present invention provides a urine sample collector for women comprising: a closed bottom; a side wall having an upper section and a lower section; and a handle attached on the lower section of the side wall. The lower section of the side wall is integrally attached to the rim of the closed bottom to form a vessel for receiving and accommodating a urine sample. The upper section of the side wall gradually flares to form a top opening which has a generally elliptical shape more elongated toward a first end than toward a second end opposite to the first end. The upper section connecting to the first end is taller and flares more outwardly than the upper section connecting to the second end. Therefore, the top opening forms a shape to fit a female genital region. The handle is provided directly below the first end, by which a user, either right-handed or left-handed, can hold the urine sample collector from the front side of her body.

### FIELD OF THE INVENTION

The present invention relates to a urine sample collector that is particularly useful in collecting urine samples from women at home, hospitals, or medical offices.

### BACKGROUND OF THE INVENTION

The conventional urine sample collector is basically a disposable cup, made of paper, styrofoam, or plastic. The opening of the cup is circular with a diameter of roughly 6-8 cm, unsuited for receiving the generally irregular urine flow emitted from the female genital region. As a result, spills are likely to occur, soiling the hands, thighs, clothes, etc. of a female user as well as the toilet seat. To clean up the mess, she would need to use more toilet paper than usual, giving rise to waste of resources. This is not only an unsanitary situation, but also an embarrassing, shameful, and awkward situation that generally confronts female users in collecting urine samples.

A variety of urine collecting devices have been devised in the past to facilitate urine collection, particularly for women. The following summarizes some of the proposed devices.

Both U.S. Pat. Nos. 6,775,852 and 6,358,477 disclose a device comprising a flexible sheet for attaching an edge to a toilet seat to form a funnel within the bowl of the toilet and a collection cup attached at the bottom of the funnel. Although this structure allows a user to avoid directly holding the cup during passing of urine, the additional steps involved in using this type of device, i.e., attaching the sheet to the toilet seat and removing it from the toilet seat, are tedious and may even cause unsanitary situations.

U.S. Pat. No. 4,198,979 discloses a funnel shaped urine collector which is directly affixed on a woman's genital area by inserting a section intravaginally. This type of device is useful for continuously collecting urine from a bedridden woman. U.S. Pat. No. 5,125,118 discloses a device including a conduit which is to be placed within the interior genital region of a woman for obtaining a relatively contaminant-free urine sample. However, these devices that directly contact the female interior genital region or vagina are extremely uncomfortable to wear, and could cause great pain and mental stress.

A specimen collection assembly disclosed by U.S. Pat. No. 6,719,951 simply comprises a collection cup and an elongated handle coupled to the cup for holding and supporting the cup in an appropriate position. The elongated handle needs to be made of a rather rigid material in order to provide mechanical strength for suspending the cup weighted with urine. Therefore, it is difficult to make the elongated handles disposable. Furthermore, stacking and storing the elongated handles can be difficult due to its shape and length.

Japanese Patent Application No. 11-312829 discloses a urine collection funnel wherein one opening is shaped to fit a female genital region rather than being circular, and the other opening is to be inserted into a conventional urine collection cup. Due to the shape of the one opening, spills are less likely to occur than the case of the conventional circular opening. Although a tab is provided on the side wall of the funnel, it is not meant for manual holding because if a user holds the tab attached on the funnel, the cup weighted with urine will fall. In addition, the tab is provided at the location where manual holding from the front side of the user's body is not possible. The tab seems to be meant for maintaining the funnel in position with the cup. Therefore, with this urine collection funnel, manual holding of the cup is expected in the conventional way so that there is still a possibility of soiling caused by the spills.

### SUMMARY OF THE INVENTION

In view of the above disadvantages inherent in prior art, the present invention provides a new urine sample collector for women which is capable of collecting urine samples sanitarily, easy to stack and store, disposable after use, and has low manufacturing cost.

According to the present invention, the urine sample collector for women comprises: a closed bottom; a side wall having an upper section and a lower section; and a handle attached on the lower section of the side wall. The lower section of the side wall is integrally attached to the rim of the closed bottom to form a vessel for receiving and accommodating a urine sample. The upper section of the side wall gradually flares to form a top opening which has a generally elliptical shape more elongated toward a first end than toward a second end opposite to the first end. The upper section connecting to the first end is taller and flares more outwardly than the upper section connecting to the second end. Therefore, the top opening forms a shape to fit a female genital region. The handle is provided directly below the first end, by which a user, either right-handed or left-handed, can hold the urine sample collector from the front side of her body.

It is preferable that the handle is foldable and can be folded against the side wall prior to use so that stacking of the urine sample collectors for storage is possible. It is further preferable that the handle is provided as a single flap or a pair of flaps of a two-dimensional shape such as a ring, a triangle, a rectangle, etc.

It is also preferable that the closed bottom has a circle shape or an oval shape.

It is also preferable that the urine sample collector is made of a material with a light color for enabling easy visual inspection of a urine sample. It is further preferable that the material is paper, plastic, or styrofoam.

### BRIEF DESCRIPTION OF THE DRAWINGS

FIG. 1 shows a perspective view of the urine sample collector for women. The boundary between the upper section and the lower section is indicated by the dash-dot line.

FIG. 2 shows a top view of the urine sample collector for women.

FIG. 3 shows a side view of the urine sample collector for women. The boundary between the upper section and the lower section is indicated by the dash-dot line.

FIG. 4 shows the way to stack the urine sample collectors, and the way to adjust the handle prior to use.

### DETAILED DESCRIPTION OF THE PREFERRED EMBODIMENTS

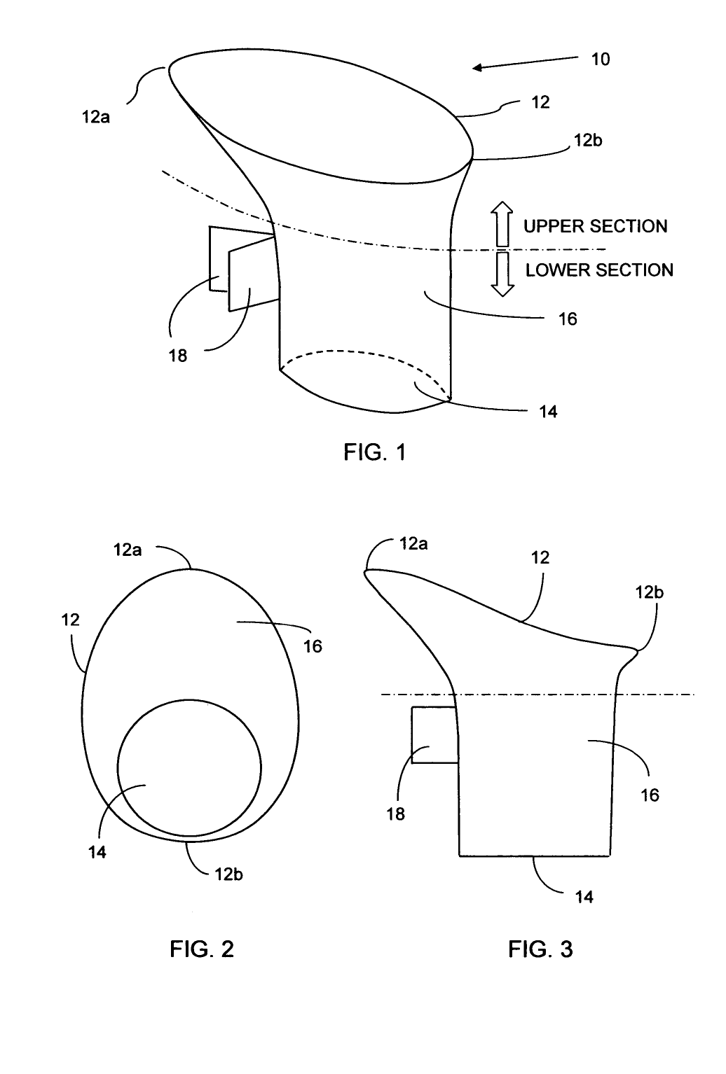

FIGS. 1, 2, and 3 show a perspective view, a top view, and a side view, respectively, of a urine sample collector for women 10 according to the present invention. The collector 10 has a top opening 12, a closed bottom 14, a side wall 16, and a handle 18. The top opening 12 has a generally elliptical shape more elongated toward one end 12a than toward the opposite end 12b as shown in FIG. 2. The closed bottom 14 and the lower section of the side wall 16 form a vessel to receive and accommodate a urine sample; the upper section of the side wall 16 gradually flares to form the above described shape of the top opening 12. As can be seen clearly in FIG. 3, the upper section of the side wall 16 connecting to the elongated end 12a is taller and flares more outwardly than the upper section of the side wall 16 connecting to the opposite end 12b. The handle 18 is foldably attached on the lower section of the side wall 16, directly below the elongated end 12a.

As shown in FIGS. 1-3, the top opening 12 is shaped to fit the female genital region rather than being circular as in a conventional urine collection cup. Thus, the urine sample collector according to the present invention is capable of receiving the generally irregular urine flow emitted from the female genital region, giving rise to less spills than in the case of a conventional urine collection cup.

The handle 18 enables a user to easily hold the collector 10. Both a right-handed user and a left-handed user can have a similar ease of holding from the front side of their body by use of the handle 18 because of the symmetrical shape of the collector 10. Since the handle 18 is provided directly below the elongated end 12a, under the flared upper section of the side wall 16, the user's hand is less likely to get soiled even if there are some spills.

The shape of the vessel can be that of a cup, a container with an oval shaped bottom, or any other as long as it serves to receive and accommodate a urine sample.

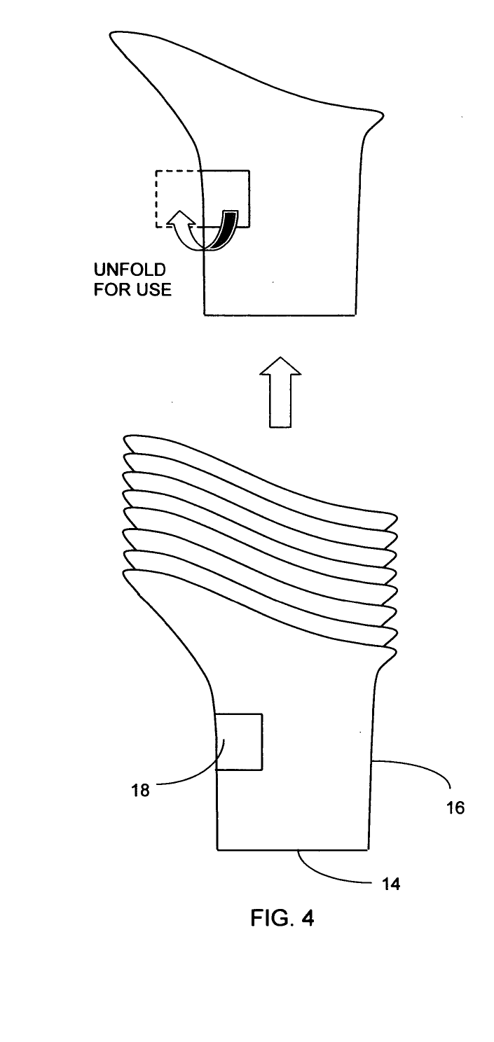

FIG. 4 shows the easy stacking of the urine sample collectors 10 for storage. This is realized by folding the handle 18 against the side wall 16 so that the handles are not in the way for stacking. A user unfolds the handle 18 to an appropriate position for holding prior to use. In FIGS. 1-4, the handle 18 is shown as a pair of flaps. However, the shape of the handle 18 may be of any shape as long as it is foldable against the side wall 16 and easy for a user to unfold and hold, such as a two-dimensional ring, a triangle, a rectangle, etc. provided as a single or a pair.

The urine sample collector 10 has a simple construction so that it can be made of any material as long as the material is durable enough for holding urine for a short time and flexible enough to form the shape, such as paper, styrofoam, or plastic. The color of the material may be any color as long as it does not interfere with the visual inspection of urine. Therefore, the urine sample collector according to the present invention can be made disposable due to low manufacturing cost.

It is to be understood that the above-described embodiments are illustrative of only a few of the many possible specific embodiments which can represent applications of the principles of the invention. Numerous and varied other arrangements can be readily devised by those skilled in the art without departing from the spirit and scope of the invention.

1. A urine sample collector for women, comprising:
a closed bottom having a rim;
a side wall having an upper section and a lower section, wherein
the lower section is integrally attached to the rim of the closed bottom to form a vessel for receiving and accommodating a urine sample, and
the upper section gradually flares to form a top opening which has a generally elliptical shape more elongated toward a first end than toward a second end opposite to the first end, the upper section connecting to the first end being taller and flaring outwardly more than the upper section connecting to the second end, whereby the top opening forms a shape to fit a female genital region; and
a handle attached on the lower section of the side wall, directly below the first end, by which a user, either right-handed or left-handed, can hold the urine sample collector from the front side of her body.

2. The urine sample collector for women as set forth in claim 1, wherein
the handle is foldable, and it is folded against the side wall prior to use so that stacking of the urine sample collectors for storage is possible.

3. The urine sample collector for women as set forth in claim 2, wherein
the handle is provided as a single flap or a pair of flaps of a two-dimensional shape.

4. The urine sample collector for women as set forth in claim 1, wherein
the closed bottom has a circular shape.

5. The urine sample collector for women as set forth in claim 1, wherein
the closed bottom has an oval shape.

6. The urine sample collector for women as set forth in claim 1, wherein
the urine sample collector is made of a material with a light color for enabling easy visual inspection of a urine sample.

7. The urine sample collector for women as set forth in claim 6, wherein
the material is paper, plastic, or styrofoam.
